(19) United States
(12) Patent Application Publication
(10) Pub. No.: US 2026/0118366 A1
(43) Pub. Date: Apr. 30, 2026

(54) METHOD FOR SELECTING OR SCREENING ANTIBODIES AND ANTIGENS FROM AN ANTIBODY LIBRARY

(71) Applicant: NEW/ERA/MABS GMBH, Potsdam (DE)

(72) Inventors: Katja Hanack, Berlin (DE); Martin Listek, Berlin (DE); Anja Schlör, Potsdam (DE)

(21) Appl. No.: 18/690,222

(22) PCT Filed: Sep. 7, 2022
(86) PCT No.: PCT/EP2022/074925

§ 371 Date: Mar. 7, 2024

(30) Foreign Application Priority Data
Sep. 7, 2021 (EP) 21195411.0

(51) Int. Cl. G01N 33/68; C40B 30/04; C40B 50/06

(52) U.S. Cl. CPC G01N 33/6845; C40B 30/04; C40B 50/06

(57) ABSTRACT

The invention relates to a new method for selecting or screening antibodies and antigens from an antibody library together with suitable expression vectors and their use.

The invention relates to a new method for selecting or screening antibodies and antigens from an antibody library together with suitable expression vectors and their use.

Monoclonal antibodies are usually obtained using the hybridoma technique, whereby antibody-producing cells (B cells or B lymphocytes) fuse with myeloma cells (cancer cells), also using fusion cell lines, resulting in hybrids that produce monoclonal antibodies (G. Köhler, C. Milstein: Continuous cultures of fused cells secreting antibody of predefined specificity. In: Nature. Vol. 256, 1975, pp. 495-497). The identification of the hybridoma cell which produces an antibody directed against the antigen can be carried out particularly advantageously with the SELMA® technology of the applicant, whereby an antibody (Ak) successfully secreted from the hybridoma cell is captured by this hybridoma cell via an Ak capture matrix or an antigen or bound to the cell surface and is identified, for example, by means of fluorescence-labeled detection antibodies.

The SELMA® technology is described in WO2015161835. In particular, the antibody-producing hybridoma cells are modified by using artificial surface markers. This creates a direct link between the antibody phenotype and the genotype of the hybridoma cell. In this way, hybridoma cells that produce the desired antibodies can be enriched extremely efficiently and in a time-saving manner using MACS or FACS technology.

WO 2015161835 A1 also describes the use of a ligation peptide sequence with the function that the binding of an adapter ligand can take place, such as avidin/biotin. The released antibody from the hybridoma cell binds to the adapter ligand via an antigen-(strept)avidin complex with an epitope, whereby the antibody binds to the epitope of the antigen and can in turn be assigned to the hybridoma cell and sorted out via the (strept)avidin-biotin binding (adapter ligand). Alternatively, the antigen can be replaced with an antibody capture matrix.

With the help of flow cytometry, for example, such fluorescence-labeled constructs can be sorted and isolated using a flow sorter or FACS (fluorescence-activated cell sorting).

However, WO 2015161835 A1 does not disclose antibody libraries and a selection of antibodies therefrom. Furthermore, WO 2015161835 A1 does not disclose the screening of antibodies using one or more antigens.

Antibodies in the prior art can be generated using phage display methods (e.g. WO91/17271, WO92/001047, WO92/20791) or selected from a human combinatorial monoclonal antibody library (e.g. HuCAL® from Morphosys®). Human antibodies can also be produced using transgenic animals carrying a human immunoglobulin gene (e.g. WO93/12227). Large libraries of fully or partially synthesized antibody combining sites or paratopes have been produced using filamentous phage display vectors called phagemids, which have resulted in large libraries of monoclonal antibodies with diverse and novel immune specificities. The technology utilizes a filamentous phage coat protein membrane anchor domain as a means to link the gene product and the gene during the assembly stage of filamentous phage replication and has been used to clone and express antibodies from combinatorial libraries.

For example, a phage display can be provided as follows:

Phage display Antibody-producing B cells or their mRNA are isolated from human blood, for example. After producing a cDNA, the DNA fragments coding for the variable regions (V_{L}, V_{H}) of the light and heavy chain genes are amplified using PCR (polymerase chain reaction). In a second PCR, V_{L} and V_{H} fragments are joined together via a linker sequence that codes for the scFv fragment. The resulting fragments code for V_{H}-G-G-G-G-G-S-S-S-V_{L} sequences (“AK”). Using phagemid vectors, for example, the PCR fragments can be bound to a DNA fragment consisting of a truncated gene coding for the minor coat protein pIII of the M13 phage and a gene coding for a signal peptide and expressed in E. coli. The signal peptide causes the fusion proteins to enter the periplasm. After coinfection with an M13 helper phage, which allows the formation of the unmodified pIII and other phage proteins, the fused “AK” are integrated into the outer envelope of the phage. The phages contain the genetic information of the respective “AK” protein, which they present on their surface. The method by which the phages that present the “correct” “AK” protein can be recognized via its antigen-binding capabilities is called “biopanning”. In this way, the DNA sequence of the DNA fragments coding for the variable regions of the human light and human heavy chains of a particular antigen-binding AK are obtained, which are subsequently joined with DNA coding for a human Fc part of an “AK” to obtain a complete gene coding for an “AK”. However, the process is complex and time-consuming. Alternative displays such as ribosome display are described in the prior art.

Such antibody libraries are the subject of the present invention. In the prior art, an antibody library can be used to screen for a target, in particular antigen, and the binders (antibody-antigen product) are selected using an enzyme-linked immunosorbent assay. The antibody genes must then be isolated and cloned in order to produce the identified antibody fragment. Since the phage display usually contains the variable part of the antibody, a suitable Fc part must be added for the production of a full-length peptide.

The disadvantages of this solution in the state of the art lie in the extensive work steps, such as a considerable cloning effort for the transfer of suitable antibody candidates to antibody production. Each antibody candidate must be cloned individually. If the antibodies are not functional after production and purification, the entire process including phage display must be repeated.

The task is therefore to improve the complex screening procedure.

Surprisingly, by integrating an antibody library into an expression vector for the selection of screened antibodies by means of an antigen, production can be carried out directly, in particular advantageously of a full-length format, since the identified antibody (or parts thereof) directly indicates the production cell. Furthermore, it is surprising that the use of several antigens is possible at the same time and screening can be carried out, although the secretion of the various antibodies into the culture medium can result in (foreign) loading by non-specific antigens. This also applies to the surface protein according to the invention containing a ligation peptide sequence. It is therefore particularly surprising that the method according to the invention can be successfully carried out simultaneously for different secreted antibodies from even different antibody libraries.

The problem is therefore solved by the technical teaching of at least one patent claim.

Therefore, the invention relates to a method for selecting or screening one or more antibodies from one or more antibody libraries, wherein at least one polynucleotide stably integrated into the genome after its transformation is used in an expression vector in a host cell, wherein the expression vector comprises at least:
i.) Polynucleotide coding for at least one antibody library,
ii.) polynucleotide encoding a surface protein comprising a ligation peptide sequence and/or at least one epitope for at least one antibody from one or more antibody libraries,
wherein
a.) the antibodies of the antibody library secrete from the host cell into the culture medium and bind to at least one added antigen comprising an adapter, wherein the adapter binds to the ligation peptide sequence of ii.),
and/or
b.) the antibodies of the antibody library secrete from the host cell into the culture medium and bind to at least one added antigen, wherein the secreted antibody binds to at least one epitope of ii.).

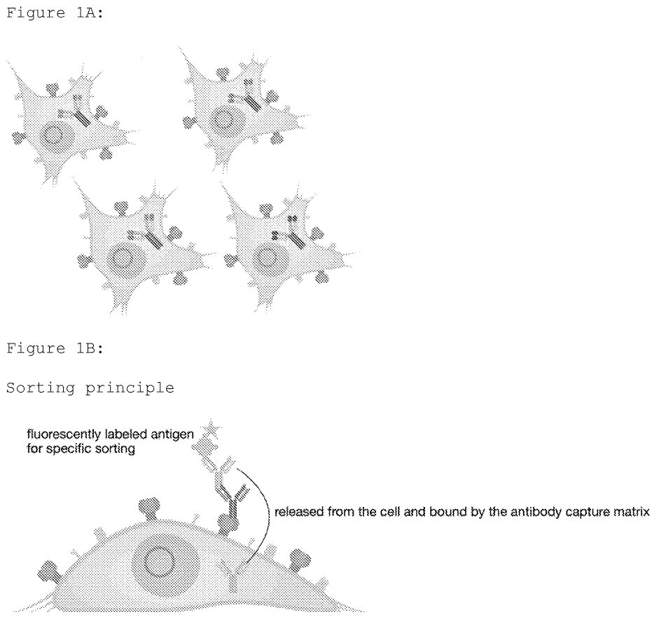

Such an initial situation of antibody libraries provided according to the invention is shown in FIG. 1A.

For example, the Fc part of the secreted antibody can bind to an epitope from b.).

In a further preferred embodiment, the bound antigen and/or the bound antibody from a.) or b.) can be labeled using conventional methods, for example with labeled (secondary) antibodies (e.g. fluorescence) or magnetic beads.

Using flow cytometry, for example, such fluorescence-labeled constructs as shown in FIG. 1B (antibody capture matrix) or an antigen can be sorted and isolated using a flow sorter or FACS (fluorescence-activated cell sorting).

The at least one added antigen can be used to screen for a suitable antibody from the antibody library and the antibody can be produced directly, e.g. after cell sorting.

Therefore, the invention relates to the use of at least one or more than one antigen for selecting or screening one or more antibodies from one or more antibody libraries, wherein at least one polynucleotide stably integrated into the genome after its transformation is used in an expression vector in a host cell, wherein the expression vector comprises at least:
i.) Polynucleotide coding for at least one antibody library,
ii.) polynucleotide encoding a surface protein comprising a ligation peptide sequence and/or at least one epitope for at least one antibody from one or more antibody libraries,
wherein
a.) the antibodies of the antibody library secrete from the host cell into the culture medium and bind to at least one added antigen comprising an adapter, wherein the adapter binds to the ligation peptide sequence of ii.),
and/or
b.) the antibodies of the antibody library secrete from the host cell into the culture medium and bind to at least one added antigen, wherein the secreted antibody binds to at least one epitope of ii.).

The “at least one antigen” can be of any nature and can in particular be a chemical substance, protein, peptide, active ingredient, drug or antibody. “At least one antigen” means that the antigens are the same or preferably different in terms of chemical composition and respective amount or concentration. For example, more than one antigen, in particular 100 or 1000 or more different antigens can be used.

Surprisingly, the method according to the invention is sufficiently robust to cope with this multitude of antibodies and antigens and to enable a selection or screening of antibodies to antigens and vice versa.

The said epitope is the object of the surface protein, in particular the epitope can be formed as part of an antibody or an antigen.

The host cell is particularly preferably a mammalian cell including a human cell, in particular selected from HEK293 cells, CHO cells, in particular cell lines, such as, not exhaustively, NS0, Sp2/0, PER.C6, BHK, COS-7 and others. Suitable cells for antibody production are known to the skilled person (Kunert R, Reinhart D. Advances in recombinant antibody manufacturing. Appl Microbiol Biotechnol. 2016 April; 100(8):3451-61. doi: 10.1007/s00253-016-7388-9).

In the case of biotin, said ligation peptide sequence has a biotin acceptor peptide sequence or biotinylation peptide, such as preferably GLNDIFEAQKIEWHE (SEQ ID No. 1) or LNDIFEAQKIEWH (SEQ ID No. 2). Other biotin acceptor peptide sequences or biotinylation peptides may also be suitable, such as an alignment with 13 amino acids 1-LXXIXXXXXXXX XXX-13 according to SEQ ID. No. 3 (see D. Beckett, E. Kovaleva, and P. J. Schatz, A minimal peptide substrate in biotin holoenzyme synthetase-catalyzed biotinylation, Protein Sci. 1999 April; 8 (4): 921-929), where:
amino acid 2 can be N, C or any or removed,
amino acid 3 can be any, except L, V, I, W, F, Y,
amino acid can be 5 F or L,
can be amino acid 6 E or D,
Amino acid 7 can be A, G, S, T,
can be amino acid 8 Q or M,
Amino acid 10 can be I, M, V,
amino acid 11 can be E, L, V, Y, I,
amino acid 12 can be W, Y, V, F, L, I,
Amino acid 13 R, H or any, but excluding D, E.

### FIG. 1: Biotinylation of AviTag (Biotinylation Peptide) Using BirA (Biotin Protein Ligase)

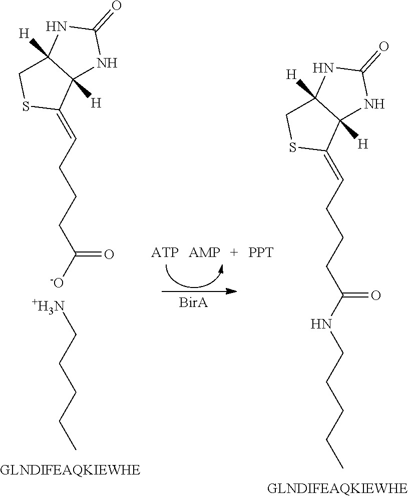

In a preferred embodiment of the invention, the expression vector comprises polynucleotides encoding at least one biotin protein ligase (BirA). Particularly advantageously, according to the invention, biotin-protein ligase (BirA) is released intracellularly. The biotinylation of a biotinylation peptide takes place intracellularly, preferably in the endoplasmic reticulum. Subsequently, the biotinylated receptor or the biotinylation peptide comprising biotin is transported to the cell surface in the form of a surface protein comprising a biotinylation peptide which binds and presents biotin. In a further preferred embodiment, BirA comprises an endoplasmic reticulum retention signal such as KDEL (SEQ ID No. 4).

The added antigen has an adapter, wherein the adapter binds to the biotin of the biotinylation peptide, and the bound antigen is labeled, and the adapter is selected from avidin, streptavidin, neutravidin.

In particular, the method according to the invention is carried out under natural, physiological conditions (e.g. culture medium in an incubator) in which the host cells remain in the culture medium. The vitality of the cells remains advantageously unaffected. The culture medium may contain, for example, biotin and other auxiliary substances and additives.

In a further preferred embodiment, the addition of the multiple and various antigens is carried out in the cold, such as 4 degrees Celsius, so that secretion of the antibody cannot occur. Said addition or incubation can preferably take place successively, for example at intervals of 20-40 minutes, so that, for example, after removal of the host cells, a new addition (or incubation) can take place or the antigens can re-suspend in the culture medium.

The antibodies of the antibody library can then be secreted from the host cell into the culture medium at a temperature of e.g. 37 degrees Celsius.

Therefore, the invention relates to a method wherein the addition of the multiple and various antigens is carried out in the cold and subsequently the antibodies of the antibody library are secreted from the host cell into the culture medium in the heat.

In a further advantageous process step, blocking agents such as free avidin, streptavidin, neutravidin on biotin of the biotinylation peptide can be used to avoid unspecific binding. Therefore, the invention relates to a method wherein blocking agents are additionally added after addition of the at least one antigen to the culture medium.

In a preferred embodiment, the expression vector according to the invention contains the following further features, such as: Signal sequence, HA tag (SEQ ID No. 5, SEQ ID No. 6), DNA biotinylation sequence (SEQ ID No. 7), EGFR signal sequence (e.g. SEQ ID No. 8, SEQ ID No. 9), EGFR sequence (SEQ ID No. 10, SEQ ID No. 11 incl. transmembrane domain), IRES (SEQ ID No. 12) as well as birA sequence (SEQ ID No. 13 incl. retention signal).

The indicated sequences include polynucleotides as well as the expressed and translated sequences.

Preferred suitable promoters include, but are not limited to, EF-1, SV-40, CMV, CAG, and many others, including conditionable promoters such as TET on, off, light-dependent promoters or stress-inducible promoters (e.g. temperature) as well as with the aid of auxiliary sequences such as IRES (internal ribosome entry site) 2A peptides and others. Such promoters and auxiliary sequences are known to the person skilled in the art.

The surface protein according to the invention can, for example, be safely transported to the cell surface with the aid of an epidermal growth factor receptor (EGFR (supra), ErbB-1, HER1, etc.) and represented on the cell surface. Furthermore, the surface protein can have a control epitope, such as hemagglutinin A epitope YPYDVPDYA (SEQ ID No. 14), so that it can be determined by cytometry or fluorescence microscopy with the aid of an antibody whether the surface protein is present extracellularly on the cell membrane.

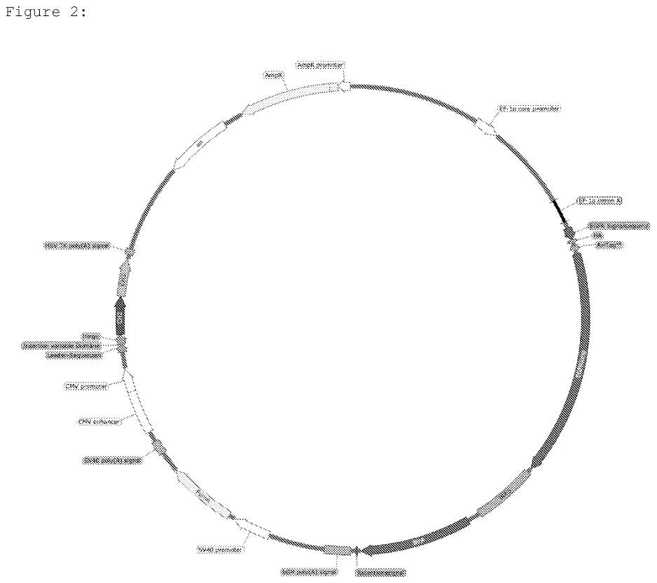

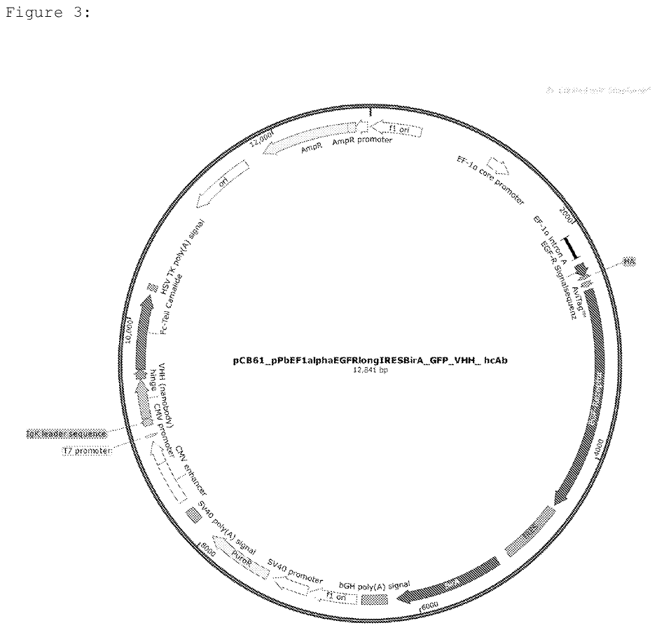

The functional units (vector map) of an expression vector according to the invention are shown by way of example in FIGS. 2 and 3.

An example of a suitable expression vector is shown in FIG. 3 (SEQ ID No. 15).

Of course, the expression vector can be divided into two or more units. The person skilled in the art is able to produce corresponding expression vectors and to introduce these preferably together—simultaneously or successively—into the host cell, preferably a mammalian cell or human cell.

In a further embodiment, a dual system consisting of a donor and helper plasmid can also be used, in which the transposase is encoded on a helper plasmid. The donor plasmid contains a cassette that is integrated into the genome. It is also possible for the transposase and the donor construct to be encoded on a vector.

The term “antibody” as used in the present invention relates to immunoglobulin molecules or immunologically active portions of immunoglobulin molecules, i.e., molecules containing an antibody-combining site or paratope. Exemplary antibody molecules include intact immunoglobulin molecules, substantially intact immunoglobulin molecules and portions of an immunoglobulin molecule, including those portions referred to in the art as Fab, Fab′, F(ab′)2 and F (v).

An antibody is therefore a protein with one or more polypeptides encoded by immunoglobulin genes or polynucleotides that specifically bind an antigen on at least one epitope. The known immunoglobulin genes include the kappa, lambda, alpha (IgA), gamma (IgG1, IgG2, IgG3, IgG4), delta (IgD), epsilon (IgE) and mu (IgM) genes of the constant region as well as the countless immunoglobulin genes of the variable region. Light immunoglobulin chains of full length are usually about 25 kD or 214 amino acids long. Full-length immunoglobulin heavy chains are usually about 50 kD or 446 amino acids long. Light chains are encoded by a variable region gene at the NH: terminus (approximately 110 amino acids long) and by a kappa or lambda constant region gene at the COOH terminus. Heavy chains are encoded in a similar way by a gene of the variable region (approx. 116 amino acids long) and by a gene of the other constant region.

The basic structural unit of an antibody is usually a tetramer consisting of two identical pairs of immunoglobulin chains, each pair containing a light and a heavy chain. In each pair, the variable domains of the light and heavy chains bind to an antigen and the constant domains mediate effector functions. Immunoglobulins also occur in a variety of other forms, including, for example, Fv, Fab, and (Fab′)2, as well as bifunctional hybrid antibodies and single chains. A variable region of an immunoglobulin light or heavy chain comprises a frame region interrupted by three hypervariable regions, also known as complementarity determining regions (CDRs). As explained, the CDRs are primarily responsible for binding to an epitope of an antigen. An immune complex is an antibody such as a monoclonal antibody, chimeric antibody, humanized antibody or human antibody or a functional antibody fragment that is specifically bound to the antigen. Chimeric antibodies are antibodies whose light and heavy chain genes have been produced from immunoglobulin genes of the variable and constant region from different species (e.g. mouse, human, etc.), usually by genetic engineering.

The term “antibody binding site” encompasses the structural part of an antibody molecule consisting of variable and hypervariable regions of heavy and light chains that specifically bind an antigen and thus describe an immune response, whereby the so-called paratope of the antibody binds to the so-called epitope of the antigen.

The term “immune response” means a specific binding between a molecule containing an antigenic determinant and a molecule containing an antibody binding site, such as a total or partial antibody molecule.

Also included according to the invention are camelid antibodies which have no light chains and are therefore able to realize the antigen binding or immune reaction with only one chain.

Furthermore, the invention includes antibodies that can be produced recombinantly, in particular monoclonal antibodies, in the case of camelid antibodies, so-called nanobodies or VHH or VH fragments (Aline Desmyter, Silvia Spinelli, Alain Roussel, Christian Cambillau, Camelid nanobodies: killing two birds with one stone, Current Opinion in Structural Biology, Volume 32, 2015, Pages 1-8, ISSN 0959-440X, https://doi.org/10.1016/j.sbi.2015.01.001.).

For the purposes of the present invention, an antibody library is one which provides at least two and more different antibodies, wherein at least in the expression vector according to the invention polynucleotides coding for at least one variable domain of the light and/or heavy chain and at least one constant domain. The skilled person is able to provide such antibody libraries in an expression vector as described above. Preferably, the constant domain comprises at least one Fc moiety, in particular at least one human, murine or camelid Fc. Antibody libraries are described e.g. in: Roger R. Beerli, Monika Bauer, Regula B. Buser, Myriam Gwerder, Simone Muntwiler, Patrik Maurer, Philippe Saudan, Martin F. Bachmann, Isolation of human monoclonal antibodies by mammalian cell display, Proceedings of the National Academy of Sciences September 2008, 105 (38) 14336-14341; DOI: 10.1073/pnas. 0805942105; Hoogenboom HR. Selecting and screening recombinant antibody libraries. Nat Biotechnol. 2005 September; 23 (9): 1105-16. doi: 10.1038/nbt1126. PMID: 16151404; Ledsgaard L, Kilstrup M, Karatt-Vellatt A, Mccafferty J, Laustsen AH. Basics of Antibody Phage Display Technology. Toxins (Basel). 2018 Jun. 9; 10(6):236. doi: 10.3390/toxins10060236. PMID: 29890762; PMCID: PMC6024766; Kumar R, Parray H A, Shrivastava T, Sinha S, Luthra K. Phage display antibody libraries: A robust approach for generation of recombinant human monoclonal antibodies. Int J Biol Macromol. 2019 Aug. 15; 135:907-918. doi: 10.1016/j.ijbiomac.2019.06.006. Epub 2019 Jun. 3. PMID: 31170490; Reader R H, Workman R G, Maddison B C, Gough K C. Advances in the Production and Batch Reformatting of Phage Antibody Libraries. Mol Biotechnol. 2019 November; 61(11):801-815. doi: 10.1007/s12033-019-00207-0. PMID: 31468301; PMCID: PMC6785589.

Furthermore, the antibody library according to the invention may comprise a combinatorial or mutagenized antibody library, in particular naive, immune, non-immune and semi-synthetic antibody libraries (see: Lim C C, Choong Y S, Lim T S. Cognizance of Molecular Methods for the Generation of Mutagenic Phage Display Antibody Libraries for Affinity Maturation. Int J Mol Sci. 2019 Apr. 15; 20(8):1861. doi: 10.3390/ijms20081861. PMID: 30991723; PMCID: PMC6515083; Ponsel D, Neugebauer J, Ladetzki-Baehs K, Tissot K. High affinity, developability and functional size: the holy grail of combinatorial antibody library generation. Molecules. 2011 May 3; 16(5):3675-700. doi: 10.3390/molecules16053675; McConnell A D, Do M, Neben T Y, Spasojevic V, Maclaren J, Chen A P, Altobell L 3rd, Macomber J L, Berkebile A D, Horlick R A, Bowers P M, King D J. High affinity humanized antibodies without making hybridomas; immunization paired with mammalian cell display and in vitro somatic hypermutation, PLOS One. 2012; 7(11):e49458. doi: 10.1371/journal. pone. 0049458).

For the expression of the variable domain and the constant domain, so-called protein leader sequences are used, in particular preferably such as murine Ig heavy chain V region 102 (SEQ. ID No. 16), murine Ig kappa (SEQ. ID No. 17), human proinsulin (SEQ. ID No. 18), human interleukin-2 (SEQ. ID No. 19) and human trypsinogen-2 (SEQ. ID No. 20)) (Marion E. E. Watson, Compilation of published signal sequences, Nucleic Acids Research, Volume 12, Issue 13, Jul. 11, 1984, Pages 5145-5164, https://doi.org/10.1093/nar/12.13.5145, Owji H, Nezafat N, Negahdaripour M, Hajiebrahimi A, Ghasemi Y. A comprehensive review of signal peptides: Structure, roles, and applications. Eur J Cell Biol. 2018 August; 97(6):422-441. doi: 10.1016/j.ejcb.2018.06.003, Stroud R M, Walter P. Signal sequence recognition and protein targeting. Curr Opin Struct Biol. 1999 December; 9(6):754-9. doi: 10.1016/s0959-440x(99)00040-8, Chen Y, Shanmugam S K, Dalbey R E. The Principles of Protein Targeting and Transport Across Cell Membranes. Protein J. 2019 June; 38(3):236-248. doi: 10.1007/s10930-019-09847-2.

Therefore, the invention comprises an expression vector comprising polynucleotides coding for an antibody library having at least one variable region of the light chain and/or the heavy chain and at least one constant region, in particular at least one human, murine or camelid Fc part, and at least one protein leader sequence.

The invention further comprises an expression vector comprising
i.) Polynucleotide coding for at least one antibody library,
ii.) polynucleotide encoding a surface protein comprising a ligation peptide sequence and/or at least one epitope for at least one antibody from one or more antibody libraries.

Furthermore, the expression vector may have further embodiments as described above.

Furthermore, the invention preferably relates to a mammalian cell comprising an expression vector according to the invention.

The following examples and figures are intended to explain the invention in more detail without, however, limiting it.

### EXAMPLE 1

### Cell Line and Cell Culture

Human embryonic kidney cells (Expi293F HEK) were cultured at 37° C., 125 rpm and 8% CO₂ in Expi293 Expression Medium (Thermo Fisher; Waltham, USA).

### EXAMPLE 2

Transfection of the Expi293F HEK cell line (ThermoFisher; Waltham, USA) with the antibody library and the surface marker-expressing gene construct HA-AP-EGF-R (FIG. 2) was performed in a dynamic culture. The Expi293F HEK cells were adjusted to a cell number of 150E6 cells in 50 mL Expi293 Expression Medium on the day of transfection. For the transfection of the Expi293F HEK cells, 50 μg of plasmid DNA and 8 μg of helper plasmid were mixed with Opi-MEM™ I Reduced Serum Medium and ExpiFectamine™ 293 Reagent and incubated for 5 minutes. Both preparations were then mixed and incubated for a further 20 minutes. The transfection mixture was then added dropwise to the cells. The cells were cultivated dynamically for 24 hours. The positive transfectants were then selected using selection media containing 2 μg/mL puromycin (ThermoFisher; Waltham, USA) for 7 days at 37° C., 125 rpm and 8% CO₂.

### EXAMPLE 3

### Magnetic Cell Sorting (MACS)

Positively transfected HEK293T cells were sorted by MACS technology according to the manufacturer's protocol (Miltenyi; Bergisch Gladbach, Germany). In brief, cells were harvested by centrifugation (300×g for 8 min at 4° C.) and resuspended in 200 μL MACS buffer (PBS/1% BSA; 2 mM EDTA pH 7.4) and stained with 1 μg mouse anti-hemagglutinin (HA) antibody per 1×106 cells. Staining was performed for 20 min at 4° C. After incubation, the cells were washed twice in 5 mL MACS buffer and then centrifuged (300×g for 8 min at 4° C.). The cell pellet was resuspended in 200 μL MACS buffer with 10 μL of an anti-mouse IgG micro-bead suspension and incubated for a further 20 min at 4° C. After washing the cells twice in 5 mL MACS buffer, the cell pellet was resuspended in 3 mL MACS buffer and applied to a MACS LS column for magnetic cell separation. Positive cells were eluted by adding 5 mL of MACS buffer to the column after the column was removed from the magnetic field. The positive cells were centrifuged and the cell pellet was resuspended in Expi293 Expression Medium supplemented with 2 μg/mL puromycin. The resuspended cells were cultured in 125 mL culture flasks (TPP, place) at 37° C., 125 rpm and 8% CO₂.

### EXAMPLE 4

### Nanobody Library

For the production of a nanobody library, peripheral blood mononuclear cells (PBMCs) were first isolated from the whole blood of llamas (Llama glama). RNA was isolated from these PBMCs using the standard phenol-chloroform protocol (RNAPure, VWR). The complementary DNA (CDNA) was then generated using reverse transcriptase. This serves as the basis for the amplification of the VH and VHH genes (=initial PCR). In a second PCR, if desired, the VH and VHH gene amplificates were discriminated. In the next step, the VHs and/or VHHs are integrated into the previously linearized target vector via homologous recombination. The resulting vectors represent the nanobody library.

### EXAMPLE 5

### Fluorescence Microscopy of Positive Transfectants

For immunofluorescence, 3×105 HA-AP-EGF-R+ HEK293T cells were seeded on poly-L-lysine-coated glass slides in 6-well plates and incubated in DMEM complete medium supplemented with 2 μg/mL puromycin at 37° C. and 8% CO-overnight. The cells were then washed with sterile PBS and fixed with 4% paraformaldehyde in PBS for 20 min at RT. After fixation, slides were washed with sterile PBS and soaked with PBS/1% BSA (20 min at RT) to avoid non-specific binding events. Phycoerythrin (PE)-conjugated streptavidin was used to detect the biotinylated surface receptor. For this purpose, 2 μg of PE-streptavidin conjugate (eBioScience 12-4317; 1E6 cells) was diluted in PBS/1% BSA and incubated for 20 min. Afterwards, a washing step with PBS/1% BSA was performed. For fluorescence microscopy analysis, slides were washed again with sterile PBS and once with distilled water before mounting in TissueTek. The microscopic images were taken with the LSM800 (Zeiss; Oberkochen, Germany).

### EXAMPLE 6

### Flow Cytometric Analysis

For the flow cytometric analyses, the cells were washed twice in FACS buffer (PBS with 0.5% BSA and 0.01% sodium azide). Subsequently, the biotinylated surface receptor was detected by a phycoerythrin (PE)-conjugated streptavidin. For this purpose, 1 μg of the PE-streptavidin conjugate (eBioScience 12-4317; 1E6 cells) was diluted in FACS buffer and incubated for 20 min. Excess PE-streptavidin conjugate was removed by the subsequent washing step. The cells were resuspended in FACS buffer and immediately measured in a flow cytometer (BD FACSAria™ III; Franklin Lakes, USA).

1. A method for selecting or screening one or more antibodies from one or more antibody libraries, wherein at least one polynucleotide stably integrated into the genome after its transformation is used in an expression vector in a host cell, wherein the expression vector comprises at least:
i) polynucleotide encoding at least one antibody library,
ii) polynucleotide encoding a surface protein containing a ligation peptide sequence and/or at least one epitope for at least one antibody from one or more antibody libraries, wherein
a) the antibodies of the antibody library secrete from the host cell into the culture medium and bind to at least one added antigen comprising an adapter, wherein the adapter binds to the ligation peptide sequence of ii),
and/or
b) the antibodies of the antibody library secrete from the host cell into the culture medium and bind to at least one added antigen, wherein the secreted antibody binds to at least one epitope of ii).

2. The method for selecting or screening one or more antibodies from one or more antibody libraries according to claim 1, wherein labeling of the bound antigen from a or b and/or the bound antibody from a) or b) is carried out, in particular with a labeled antibody or magnetic bead and sorted and isolated with a flow sorter or fluorescence-activated cell sorting (FACS).

3. The method for selecting or screening one or more antibodies from one or more antibody libraries according to claim 1, wherein the antibody library comprises at least one variable region of the light and/or heavy chain and at least one constant region, in particular at least one human, murine or camelid Fc moiety, and at least one protein leader sequence.

4. The method for selecting or screening one or more antibodies from one or more antibody libraries according to claim 1, wherein the antibody library is selected from the group consisting of combinatorial or mutagenized antibody libraries, in particular naive, immune, non-immune and semi-synthetic antibody libraries.

5. The method for selecting or screening one or more antibodies from one or more antibody libraries according to claim 1, wherein the host cell is a mammalian cell, in particular HEK293 cells, CHO cells.

6. The method for selecting or screening one or more antibodies from one or more antibody libraries according to claim 1, wherein the ligation peptide sequence is a biotinylation peptide selected from the group consisting of 1-LXXIXXXXXXXX XXX-13 (SEQ ID NO: 3) wherein
amino acid 2 may be N, C or any or removed,
amino acid 3 may be any but excluding L, V, I, W, F, Y,
amino acid 5 may be F or L,
amino acid 6 may be E or D,
amino acid 7 may be A, G, S, T,
amino acid 8 may be Q or M,
amino acid 10 may be I, M, V,
amino acid 11 may be E, L, V, Y, I,
amino acid 12 may be W, Y, V, F, L, I,
amino acid 13 may be R, H or any, but excluding D, E; or
the biotinylation peptide is selected from the group consisting of GLNDIFEAQKIEWHE (SEQ ID NO: 1) and LNDIFEAQKIEWH (SEQ ID NO: 2) and the adapter is selected from the group consisting of avidin, streptavidin, neutravidin and combinations thereof.

7. The method for selecting or screening one or more antibodies from one or more antibody libraries according to claim 1, wherein the expression vector comprises polynucleotides selected from the group consisting of coding for at least one biotin protein ligase (BirA), signal sequence, HA tag (SEQ ID NO: 5, SEQ ID NO: 6), DNA biotinylation sequence (SEQ ID NO: 7), EGFR signal sequence (SEQ ID NO: 8, SEQ ID NO: 9), EGFR sequence (SEQ ID NO: 10, SEQ ID NO: 11 including a transmembrane domain), IRES (SEQ ID NO: 12), birA sequence (SEQ ID NO: 13 including a retention signal), and combinations thereof.

8. The method for selecting or screening one or more antibodies from one or more antibody libraries according to claim 1, wherein the addition of the multiple and different antigens is carried out in the cold and subsequently in the heat the antibodies of the antibody library secrete from the host cell into the culture medium.

9. The method for selecting or screening one or more antibodies from one or more antibody libraries according to claim 1, wherein blocking agents are additionally added after addition of the at least one antigen to the culture medium.

10. Expression An expression vector comprising
i) polynucleotide encoding at least one antibody library,
ii) a polynucleotide encoding a surface protein containing a ligation peptide sequence and/or at least one epitope for at least one antibody from one or more antibody libraries.

11. A mammalian cell comprising the expression vector according to claim 10.

12. A method of using at least one or more than one antigen for selecting or screening one or more antibodies from one or more antibody libraries, wherein at least one polynucleotide stably integrated into the genome after its transformation is used in an expression vector in a host cell, wherein the expression vector comprises at least:
i) polynucleotide encoding at least one antibody library,
ii) polynucleotide encoding a surface protein containing a ligation peptide sequence and/or at least one epitope for at least one antibody from one or more antibody libraries, wherein
a) the antibodies of the antibody library secrete from the host cell into the culture medium and bind to at least one added antigen comprising
an adapter, wherein the adapter binds to the ligation peptide sequence of ii), and/or
b) the antibodies of the antibody library secrete from the host cell into the culture medium and bind to at least one added antigen, wherein the secreted antibody binds to at least one epitope of ii).


SEQUENCE LISTING (21 sequences)

SEQ ID NO: 1 - protein, 15 aa, synthetic construct
GLNDIFEAQKIEWHE

SEQ ID NO: 2 - protein, 13 aa, synthetic construct
LNDIFEAQKIEWH

SEQ ID NO: 3 - skipped

SEQ ID NO: 4 - protein, 4 aa, synthetic construct
KDEL

SEQ ID NO: 5 - DNA, 27 bp, unidentified
  source 1..27: mol_type = other DNA
tatccatatgatgttccagattatgct

SEQ ID NO: 6 - protein, 9 aa, unidentified
YPYDVPDYA

SEQ ID NO: 7 - DNA, 45 bp, synthetic construct
  source 1..45: mol_type = other DNA
ggcctgaacgacatcttcgaggcccagaagatcgagtggcacgag

SEQ ID NO: 8 - DNA, 72 bp, Homo sapiens
  source 1..72: mol_type = genomic DNA
atgcgaccctccgggacggccggggcagcgctcctggcgctgctggctgcgctctgcccggcgagtcgggct

SEQ ID NO: 9 - protein, 24 aa, Homo sapiens
MRPSGTAGAALLALLAALCPASRA

SEQ ID NO: 10 - protein, 1956 aa, Homo sapiens
CTGGAGGAAAAGAAAGTTTGCCAAGGCACGAGTAACAAGCTCACGCAGTTGGGCACTTTTGAAGATCATTTTCTCAGCCTCCAGAGGATGTTCAATAACTGTGAGGTGGTCCTTGGGAATTTGGAAATTACCTATGTGCAGAGGAATTATGATCTTTCCTTCTTAAAGACCATCCAGGAGGTGGCTGGTTATGCCCTCATTGCCCTCAACACAGTGGAGCGAATTCCTTTGGAAAACCTGCAGATCATCAGAGGAAATATGTACTACGAAAATTCCTATGCCTTAGCAGTCTTATCTAACTATGATGCAAATAAAACCGGACTGAAGGAGCTGCCCATGAGAAATTTACAGGAAATCCTGCATGGCGCCGTGCGGTTCAGCAACAACCCTGCCCTGTGCAACGTGGAGAGCATCCAGTGGCGGGACATAGTCAGCAGTGACTTTCTCAGCAACATGTCGATGGACTTCCAGAACCACCTGGGCAGCTGCCAAAAGTGTGATCCAAGCTGTCCCAATGGGAGCTGCTGGGGTGCAGGAGAGGAGAACTGCCAGAGACTGACCAAAATCATCTGTGCCCAGCAGTGCTCCGGGCGCTGCCGTGGCAAGTCCCCCAGTGACTGCTGCCACAACCAGTGTGCTGCAGGCTGCACAGGCCCCCGGGAGAGCGACTGCCTGGTCTGCCGCAAATTCCGAGACGAAGCCACGTGCAAGGACACCTGCCCCCCACTCATGCTCTACAACCCCACCACGTACCAGATGGATGTGAACCCCGAGGGCAAATACAGCTTTGGTGCCACCTGCGTGAAGAAGTGTCCCCGTAATTATGTGGTGACAGATCACGGCTCGTGCGTCCGAGCCTGTGGGGCCGACAGCTATGAGATGGAGGAAGACGGCGTCCGCAAGTGTAAGAAGTGCGAAGGGCCTTGCCGCAAAGTGTGTAACGGAATAGGTATTGGTGAATTTAAAGACTCACTCTCCATAAATGCTACGAATATTAAACACTTCAAAAACTGCACCTCCATCAGTGGCGATCTCCACATCCTGCCGGTGGCATTTAGGGGTGACTCCTTCACACATACTCCTCCTCTGGATCCACAGGAACTGGATATTCTGAAAACCGTAAAGGAAATCACAGGGTTTTTGCTGATTCAGGCTTGGCCTGAAAACAGGACGGACCTCCATGCCTTTGAGAACCTAGAAATCATACGCGGCAGGACCAAGCAACATGGTCAGTTTTCTCTTGCAGTCGTCAGCCTGAACATAACATCCTTGGGATTACGCTCCCTCAAGGAGATAAGTGATGGAGATGTGATAATTTCAGGAAACAAAAATTTGTGCTATGCAAATACAATAAACTGGAAAAAACTGTTTGGGACCTCCGGTCAGAAAACCAAAATTATAAGCAACAGAGGTGAAAACAGCTGCAAGGCCACAGGCCAGGTCTGCCATGCCTTGTGCTCCCCCGAGGGCTGCTGGGGCCCGGAGCCCAGGGACTGCGTCTCTTGCCGGAATGTCAGCCGAGGCAGGGAATGCGTGGACAAGTGCAACCTTCTGGAGGGTGAGCCAAGGGAGTTTGTGGAGAACTCTGAGTGCATACAGTGCCACCCAGAGTGCCTGCCTCAGGCCATGAACATCACCTGCACAGGACGGGGACCAGACAACTGTATCCAGTGTGCCCACTACATTGACGGCCCCCACTGCGTCAAGACCTGCCCGGCAGGAGTCATGGGAGAAAACAACACCCTGGTCTGGAAGTACGCAGACGCCGGCCATGTGTGCCACCTGTGCCATCCAAACTGCACCTACGGATGCACTGGGCCAGGTCTTGAAGGCTGTCCAACGAATGGGCCTAAGATCCCGTCCATCGCCACTGGGATGGTGGGGGCCCTCCTCTTGCTGCTGGTGGTGGCCCTGGGGATCGGCCTCTTCATGCGAAGGCGCCACATCGTTCGGTAA

SEQ ID NO: 11 - protein, 651 aa, Homo sapiens
LEEKKVCQGTSNKLTQLGTFEDHFLSLQRMFNNCEVVLGNLEITYVQRNYDLSFLKTIQEVAGYALIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQRLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVR

SEQ ID NO: 12 - DNA, 552 bp, synthetic construct
  source 1..552: mol_type = other DNA
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 13 - DNA, 1005 bp, Escherichia coli
  source 1..1005: mol_type = genomic DNA
atgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgcctgtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaagggtctacatctggatctgggggttcaaaggacgagctgtga

SEQ ID NO: 14 - protein, 9 aa, synthetic construct
YPYDVPDYA

SEQ ID NO: 15 - DNA, 12502 bp, unidentified
  source 1..12502: mol_type = unassigned DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggggcgcgccattctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaactagaatgctagcagatctcgaccgagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgatcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttgaccaagcttcgaattctgcagtcgacggtaccgccaccatgcgaccctccgggacggccggggcagcgctcctggcgctgctggctgcgctctgcccggcgagtcgggctctggaggaaggctcgtctaagcttggctcgtctgggtatccatatgatgttccagattatgctggggcccagccggccagatctggcggcggcctgaacgacatcttcgaggcccagaagatcgagtggcacgagggcgcgccggctaccggaagcagcgggctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgccctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagagactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggtaagcggccgctctagcccaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgggccgaccctccgggacggccggggcagcgctcctggcgctgctggctgcgctctgcccggcgagtcgggctctggaggaaggctcgtctaagcttggctcgtctgggatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgcctgtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaagggtctacatctggatctgggggttcaaaggacgagctgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtgggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcccgcggatcgatggatccattcatgaatgaattcatgtcgacatactagtgcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattcggcttggggatatccaccatggagacagacacactcctgctatgggtactgctgctctgggttccaggttccactggtgatgttaacgaacccaagacaccaaaaccacaaccacaaccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgctgaggtgcgaacggccaacacgaggccaaaagaggaacagttcaacagcacgtaccgcgtggtcagcgtcctgcccatccagcaccaggactggctgacggggaaggaattcaagtgcaaggtcaacaacaaagctctcccggcccccatcgagaagaccatctccaaggccaaagggcagacccgggagccgcaggtgtacgccctggccccacaccgggaagagctggccaaggacaccgtgagcgtaacatgcctggtcaaaggcttctacccacctgatatcaacgttgagtggcagaggaaccggcagccggagccagagggcacctacgccaccacgccgccacagctggacaacgacgggacctacttcctctacagcaagctctcggtgggaaagaacacgtggcagcggggagaaaccttcacctgtgtggtgatgcacgagaccctgcacaaccactacacccagaaatccatctcccagtcttaaaacacggaaggagacaataccggaaggaacccgcgctatgacggcaataaaaagacagaataaaacgcacgggtgttgggtcgtttgttcataaacgcggggttcggtccccatcaactagaatgctaactagttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagtatacgcgtatgcggccgcttaattaatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 16 - protein, 19 aa, Mus musculus
MGWSCIILFLVATATGVHS

SEQ ID NO: 17 - protein, 21 aa, Mus musculus
METDTLLLWVLLLWVPGSTGD

SEQ ID NO: 18 - protein, 24 aa, Homo sapiens
MALWMRLLPLLALLALWGPDPAAA

SEQ ID NO: 19 - protein, 20 aa, Homo sapiens
MYRMQLLSCIALSLALVTNS

SEQ ID NO: 20 - protein, 15 aa, Homo sapiens
MNLLLILTFVAAAVA

SEQ ID NO: 21 - protein, 7 aa, synthetic construct
GGGGSSS